(19) United States
(12) Patent Application Publication
(10) Pub. No.: US 2002/0144309 A1
(43) Pub. Date: Oct. 3, 2002

(54) Transgenic plants expressing MinD or MinE and an efficient method for plant chloroplast transformation and gene expression

(76) Inventors: Randy Dinkins, Lexington, KY (US); M.S. Srinivasa Reddy, Lexington, KY (US); Glenn B. Collins, Lexington, KY (US)

Correspondence Address: Teresa Stanek Rea, BURNS, DOANE, SWECKER & MATHIS, L.L.P., P.O. Box 1404, Alexandria, VA, 22313-1404, US

(21) Appl. No.: 10/067,989

(22) Filed: Feb. 8, 2002

Related U.S. Application Data
(60) Provisional application No. 60/267,488, filed on Feb. 9, 2001.

(51) Int. Cl. A01H 1/00; C12N 9/02; C12P 21/02; C12N 5/04

(52) U.S. Cl. 800/282; 435/189; 435/320.1; 435/419; 435/69.1

(57) ABSTRACT

The present invention concerns transgenic plant which contain large chloroplasts. The transgenic plant of the present invention comprises within its genome a foreign MinD or MinE gene or a foreign gene which expresses a protein which has the same functional activity as the Arabidopsis thaliana MinD or MinE protein. The present invention further concerns a method of producing the transgenic plants of the present invention which contain large chloroplasts. Finally, the present invention concerns a method of transforming the chloroplasts genome of the transgenic plants of the present invention which contain large chloroplasts.

### FIELD OF THE INVENTION

The present invention involves an improved method for chloroplast genome transformation. The method of the present invention involves the development of transgenic plants which contain large chloroplasts. The transgenic plants of the present invention comprise within their genome an exogenous Arabidopsis thaliana MinD or MinE genes or an exogenous gene which expresses a protein which has the same functional activity as the Arabidopsis thaliana MinD or MinE protein, such as a MinD or MinE genetic homolog from another plant. Use of the transgenic plants of the present invention in chloroplast genome transformation protocols is advantageous since the large size of the chloroplasts would make them easy targets for transformation.

### BACKGROUND OF THE INVENTION

In photosynthetic leaf cells of higher plants, the most conspicuous organelles are the chloroplasts, which exist in a semi-autonomous fashion within the cell, containing their own genetic system and protein synthesis machinery, but relying upon a close cooperation with the nucleo-cytoplasmic system in their development and biosynthetic activities. The chloroplast present in leaf cells is one developmental stage of this organelle. Proplastids, etioplasts, amyloplasts, and chromoplasts are other stages of this organelle. The embodiments of this invention apply to the organelle which includes Chloroplast and its developmental stages.

The most essential function of chloroplasts is the performance of the light-driven reactions of photosynthesis including fixation of carbon dioxide. However, chloroplasts carry out many other biosynthetic processes of importance to the plant cell, such as synthesis of fatty acids. In addition, the reducing power of light-activated electrons drives the reduction of nitrites (NO₂⁻) to ammonia (NH₃) in the chloroplast; this ammonia provides the plant with nitrogen required for the synthesis of amino acids compartmentalized in the chloroplast and nucleus.

Genetic transformation of the nuclear and chloroplast genomes has many benefits and potentials, one example being crop improvement. Transformation of the chloroplast genome, now common by particle bombardment, offers several advantages over nuclear transformation.

First, in our society today, there exists a general concern regarding the use of biologically engineered crops and the uncertainties surrounding their effects. Amongst most of the agronomically important species, the chloroplast is inherited maternally. Therefore, because pollen carries DNA from the nuclear genome and not the chloroplast genome, there is very low probability of pollen mediated outcrossing of the transgene into close wild relatives (Daniell et al., Nature Biotech. 165-348 (1998)).

Second, since there is no pollen transmission of the transformed gene, the danger of negatively impacting beneficial insects that utilize the pollen of crop plants is eliminated because the pollen does not express insecticidal compounds.

Third, it has been observed that chloroplast gene expression can be several folds higher compared to genes expressed with high constitutive expressing promoters in the nuclear genome. For example, expression of the Bacillus thuringiensis (Bt) protein in the chloroplast genome resulted in accumulation of 3-5% of the total soluble Bt protein in tobacco leaves (McBride et al., Bio/Tech. 13:362-365 (1995)). This level approaches the needed concentration levels that are necessary in the production of alternative compounds in plant cells, such as pharmaceutical compounds, or other natural products.

Fourth, may essential functions in plant metabolism, such as lipid synthesis and amino acid synthesis, occurs in the chloroplasts. Proteins that will affect these pathways must be directed into the chloroplast. By expressing the genes in the chloroplast, these gene products are readily available and do not need to be transported to the chloroplast from the nucleus.

Fifth, chloroplast gene expression is of a prokaryotic nature, enabling the expression of multiple genes from a single promoter or a polycistronic message. This circumvents two problems: (1) having to use multiple gene constructs with multiple promoters to avoid gene silencing; and (2) having to co-transform plants with different transgenes of interest in order to have them expressed in one plant.

Sixth, it is possible to introduce multiple copies of foreign genes into the chloroplast genome as opposed to the limited number of functional copies of a foreign gene which typically may be introduced via the nuclear genome. Additionally, plants engineered through the chloroplast genome rather than the nuclear genome also could have a significant energy advantage since synthesis and import of precursor proteins into a cell organelle are highly energy consuming and rate limiting processes.

Lastly, since transformation of the chloroplast genome is via homologous recombination, there are no problems associated with position effects in the chloroplast chromosome, or insertional mutagenesis by the transgene into other genes.

Chloroplast transformation has been shown to be feasible with several species, including tobacco, Arabidopsis, and potato (Svab et al., Proc. Natl. Acad. Sci. U.S.A. 90: 913-917 (1990); Sidorov et al., Plant J. 19:209-216 (1999)). However, chloroplast transformation is still far from routine. Problems associated with chloroplast transformation are primarily two fold. First, there are multiple chloroplasts per plant cell (10-100) and within each chloroplast there are 100-1000 DNA molecules. Thus, efficiency of chloroplast transformation is highly dependent upon multiple rounds of tissue culture selection to increase the number of total transgenic chloroplasts in each cell of the regenerating plantlet due to random assortment. Second, the choloroplast is typically a small target for transformation.

The prokaryotic origin and endosymbiotic nature of chloroplasts and mitochondria in plant cells is now an accepted hypothesis. Since chloroplasts are presumed to be of prokaryotic origin, it has been postulated that components of chloroplast division could be similar to those of bacteria. The present research results demonstrate that the MinD gene from Arabidopsis thaliana (a small cruciferous plant), which is homologous to the bacterial cell division inhibitor, disrupts normal chloroplast division when it is overexpressed in tobacco cells. This result is similar to what is found when MinD is overexpressed in wild-type E. coli (de Boer et al., Cell 56:641-649 (1989); Proc. Natl. Acad. Sci. USA 87:1129-1133 (1990)). It can be inferred from the bacterial models that the correct level of the MinD gene product is required for proper chloroplast division. The present results also demonstrate that the MinE gene from Arabidopsis thaliana, which is homologous to the bacterial MinE gene, can also disrupt normal chloroplast division when it is overexpressed in tobacco cells or in Arabidopsis thaliana cells.

Genes that regulate cell division and the topological specificity placement of cell division in E. coli are encoded by the minicell (min) locus: the MinC, MinD and MinE genes (de Boer et al., Cell 56:641-649 (1989)). Deletion or incorrect expression of the Min gene products in E. coli results in the FtsZ ring placement at the wrong location, giving rise to small mini-cells lacking chromosomes, or inhibits septation altogether, resulting in very long filamentous cells. The MinC gene product blocks cell division and is dependent on the MinD gene product for this activity. When MinD is not present, the MinC mediated cell division inhibition only functions when MinC is in excess (de Boer et al., Proc. Natl. Acad. Sci. USA 87:1129-1133 (1990)). Recently, Hu et al. (Proc. Natl. Acad. Sci. USA 96:14819-14824 (1999)) have shown that MinC interacts with FtsZ to prevent polymerization, thereby inhibiting division. The MinE protein is dependent on the presence of MinD and its expression precedes the FtsZ accumulation, thus determining the cell division location in E. coli (Raskin and de Boer, Cell 91:685-694 (1997)). The MinD gene product is a membrane GTPase that activates the MinC cell-dependent division inhibition and is required in MinE topological specificity (de Boer et al., EMBO J. 10:4371-4380 (1991); J. Bacteriol. 174:63-70 (1992)). At normal levels, MinC and MinD act in concert to block division at all potential cell division sites. MinE suppresses the MinC action at the mid-cell location, but not at the poles (Raskin and de Boer, Cell 91:685-694 (1997)). Thus, MinD acts as a cell division modulator by promoting the MinE action at the mid-cell location and acts in concert with MinC to repress cell division at other potential sites.

The Min genes are found in many, but not all, bacteria, including the photosynthetic cyanobacteria, Synnechocystis. The MinD and MinE genes are also found in the chloroplast genome of Chlorella in the same order as they are found in E. coli. A postulated homologue of the MinD gene was identified in the Arabidopsis nuclear sequence database.

In higher plants, the chloroplast genome encodes roughly 130 gene products. The nuclear genome encodes many of the proteins involved in the photosynthetic apparatus, and controls most aspects of chloroplast gene expression (Mullet, Annu. Rev. Plant Physiol. Plant Mol. Biol. 39:475-502 (1988); Leon et al., Annu. Rev. Plant Physiol. Plant Mol. Biol. 49:453-580 (1998)). Nuclear genes also control whether the apoplast develops into a mature photosynthesizing chloroplast or a chromoplast, etioplast or leucoplast (Link, in Cell culture and somatic cell genetics of plants, Vol. 7, The molecular biology of plastids and the photosynthetic apparatus (Bogorad, L. and Vasil, I., eds.), Academic Press, New York, pages 365-394 (1991); Herrmann et al., in Plant gene research, cell organelles (Herrmann, R. G., ed), Springer Verlag, N.Y., pages 276-349 (1992)). Most cells with photosynthesizing chloroplasts maintain a fairly constant number of chloroplasts based on cell volume (Pyke, Am. J. Bot. 84:1017-1027 (1997); Plant Cell 11:549-556 (1999)). Single nuclear recessive mutants of Arabidopsis thaliana have been isolated that reportedly affect accumulation and replication of chloroplasts (Pyke and Leech, Plant Physiol. 99:1005-1008 (1992)). Depending on the mutation, the lesion reportedly results in many small chloroplasts to a few large chloroplasts per cell. Results suggest a role for nuclear control of chloroplast division.

Efficiency of chloroplast transformation is highly dependent upon multiple rounds of tissue culture selection to increase the number of total transgenic chloroplasts DNA molecules and assortment of the chloroplasts in each cell of the regenerating plant. In tobacco, the most efficient system reported in the literature has been one event per bombarded plate (Svab and Maliga, 1993). Thus, there is a need in the art for an improved method of chloroplast transformation. One means of obtaining an improved method of chloroplast transformation would be to produce plants which have a few very large chloroplasts in their cells. The large chloroplasts would be easier targets for transformation. Thus, there is a need in the art for a transgenic plant which contain within its cells a few, very large chloroplasts.

### SUMMARY OF THE INVENTION

The present invention provides a vector which comprises an exogenous gene. The exogenous gene expressed by the vector of the present invention expresses a protein which effects a plant cell by allowing for expression of only one or a few large chloroplasts. Preferably, the exogenous gene is a MinD or MinE gene or an exogenous gene which encodes a protein which has the same functional activity as the Arabidopsis thaliana MinD or MinE protein. The MinD or MinE gene may be derived from a plant other than Arabidopsis thaliana. One of skill in the art, in view of what is known in the art and from the disclosure herein, could identify MinD and MinE genes from other plant species.

The present invention provides transgenic plants which contain large chloroplasts. The transgenic plants of the present invention comprise within their genome an exogenous MinD or MinE gene or an exogenous gene which encodes a protein which has the same functional activity as the Arabidopsis thaliana MinD or MinE protein.

The present invention also provides a method of producing the transgenic plants of the present invention, wherein said method comprises transforming the nuclear genome of a plant with a vector which comprises an exogenous MinD or MinE gene or an exogenous gene which encodes a protein which has the same activity as the Arabidopsis thaliana MinD or MinE protein.

The present invention further provides for a method of transforming the chloroplast genome of the transgenic plants of the present invention, wherein said method comprises producing the transgenic plants of the present invention, which have large chloroplasts, and transforming the large chloroplasts with a vector which comprises a gene of interest. The present invention also provides for a chloroplast transgenic plant produced by this method.

The present invention even further provides for a method of selecting for transgenic plants (produced by the method of the present invention) that are chloroplast transgenics but not nuclear transgenics. This method comprises crossing a chloroplast and nuclear transgenic plant produced by the method of the present invention with a wild-type plant. The plants which express the exogenous gene or genes of interest in the chloroplast genome, but do not express the exogenous gene in the nuclear genome, are then segregated out by identifying which plants have normal chloroplast size and number, and have the desired characteristic produced by the exogenous gene expressed in the chloroplast genome.

### DEFINITIONS

The term “derived from” a known gene or protein means that the gene or protein is the native known gene or protein, or is a gene or protein which is derived therefrom and has a significant amount of homology with said known gene or protein so that it has the same function as said known gene or protein. Preferably, a gene or protein derived from a known gene or protein should share at least about 80% similarity with said known gene or protein, preferably at least about 85%, and more preferably at least about 90% or 95% similarity.

In the context of the genes that are to be used in the vectors of this invention, “homologous” refers to genes whose expression results in expression products which have a combination of amino acid sequence similarity or identity (or base sequence similarity for transcript products) and functional equivalence, and are therefore homologous genes. In general such genes also have a high level of DNA sequence similarity (i.e., greater than 80% when such sequences are identified among members of the same genus, but lower when these similarities are noted across fungal genera), but are not identical. Preferred genetic homologs include those genes which are about at least 85%, 90% or 95% similar at the nucleic acid or the amino acid level. The combination of functional equivalence and sequence similarity means that if one gene is useful, e.g., for transforming the nuclear genome of a plant, which would then produce larger and fewer chloroplasts, then the homologous gene is likewise useful. In addition, identification of one such gene serves to identify a homologous gene through the same relationships as indicated above. Typically, such homologous genes are found in other plant species, especially, but not restricted to, closely related species.

Alignment programs can be used to identify conserved sequences or potential motifs across different plant species. Alignment programs can also be used to align the nucleic acid and/or protein sequences of related genes and the proteins that they encode. Preferred alignment programs include CLUSTALW, PILEUP and GAP, and would preferably be used with default parameters.

Due to the DNA sequence similarity, homologous genes are often identified by hybridizing with probes from the initially identified gene under hybridizing conditions which allow stable binding under appropriately stringent conditions (e.g., conditions which allow stable binding with at least approximately 85% or more sequence identity). Hybridization methods are known in the art and include, but are not limited to: (a) washing with 0.1× SSPE (0.62 M NaCl, 0.06 M NaH₂PO₄.H₂O, 0.075 M EDTA, pH 7.4) and 0.1% sodium dodecyl sulfate (SDS) at 50° C.; (b) washing with 50% formamide, 5× SSC (0.75 M NaCl, 0.075 M sodium citrate), 50 mM sodium phosphate (pH 6-8), 0.1% sodium pyrophosphate, 5× Denhardt's solution, sonicated salmon sperm DNA (50 μg/ml), 0.1% SDS and 10% dextran sulfate at 42° C., followed by washing at 42° C. in 0.2× SSC and 0.1% SDS; and (c) washing with of 0.5 M NaPO₄, 7% SDS at 65° C. followed by washing at 60° C. in 0.5× SSC and 0.1% SDS. High stringency hybridization conditions are those performed at about 20° C. below the melting temperature (Tₘ) of the probe. Preferred stringency is performed at about 5-10° C. below the melting temperature (Tₘ) of the probe. Additional hybridization conditions can be prepared as described in Chapter 11 of Molecular Cloning: A Laboratory Manual, 2nd Ed., ed. By Sambrook, Fritsch and Maniatis (Cold Spring Harbor Laboratory Press: 1989), or as would be known to the artisan of ordinary skill. The equivalent function of the product is then verified using appropriate biological and/or biochemical assays.

By a polynucleotide having a nucleotide sequence at least, for example, 90% “similar” to a reference nucleotide sequence encoding a polypeptide, is intended that the nucleotide sequence of the polynucleotide is identical to the reference sequence except that the polynucleotide sequence may include up to ten point mutations per each 100 nucleotides of the reference nucleotide sequence

### BRIEF DESCRIPTION OF THE DRAWINGS

FIG. 1. Comparison of the deduced Arabidopsis thaliana MinD amino acid sequence with known MinD gene products. Arabidopsis thaliana sequence mzf 18.5, GenBank AB009056; Chlorella vulgaris chloroplast minD, Swissprot Acc No. P56346; Synnechocystis PCC6803 MinD locus, Swissprot Acc. No. q55900, GB3024144; Escherichia coli, GenBank Acc. No. P18197. The underlined asterisks indicate a putative nucleotide binding region. The red indicates amino acid identity with the Arabidopsis MinD deduced amino acid sequence (in green), and those in blue indicate conserved amino acid substitutions. Alignments were done first using the Basic Local Alignment Search Tool (NCBI) and further aligned manually.

FIG. 2. Light microscopy of tobacco leaf cross-sections (A) of wild-type KY160 and (B) overexpressing AtMinD15. Bar represents 50 μm.

FIG. 3. Confocal microscopy of wild-type and AtMinD overexpressing tobacco plants. (A) Wild-type KY160 tobacco plant; (B) AtMinD line 5; (C) AtMinD line 14; 5 (D) AtMinD line 17. Each plate is a composite of 16 frames using the extended focus from the Leica TCS PowerScan™ software to give a false 3D image. All images were using a 100× objective lens 512×512 pixels (x-y axis) which equals 100 μm. The z axis was composed of 16 frames equally spaced through (A) 52.5 μm, (B) 31.5 μm, (C) 49.5 μm and (D) 31.5 μm through the abaxial leaf surface. Bar represents 10 μm.

FIG. 4. Transmission electron microscopy of wild-type tobacco and AtMinD tobacco overexpressing plants. (A) Wild-type KY160, (B) AtMinD line 15, (C) AtMinD line 1 and (D) AtMinD line 6. Bar represents 1 μm (A, C and D) and 2 μm (B).

FIG. 5. Southern blot of wild-type and AtMinD tobacco overexpressing plants. Wild-type KY160 lanes indicated by the “w” and each number corresponds to the AtMinD tobacco plant described in the text. Probes indicated are the tobacco rbcL gene (PCR amplified from genomic DNA) and Arabidopsis thaliana MinD gene (described in the specification.)

FIG. 6. RNA blot of wild-type and AtMinD overexpressing tobacco plants. Wild-type KY160 lanes are indicated by the “W” and each number corresponds to the AtMinD tobacco plant described in the text. Probes indicated are, minD: the Arabidopsis thaliana MinD gene described in the specification; cab8: a 550 bp internal HindIII fragment of the tomato photosystem I chlorophyll a/b binding protein gene 8 (Schwarz and Pickersky, Plant Mol. Biol. 15:157-160 (1990)); cab10B: a 700 bp internal PstI-XbaI fragment of the tomato photosystem II chlorophyll a/b binding protein (CP24) gene 10B (Pichersky et al., Plant Mol. Biol. 12:257-270 (1989)); rbcS: the small subunit of the spinach ribulose bisphosphate carboxylase (gift of Robert Houtz, University of Kentucky, USA); 28S: 28S ribosomal gene from maize; rbcL: PCR amplified large subunit of ribulose bisphosphate carboxylase from tobacco chloroplast; psbA: PCR amplified photosystem II subunit D 1 from tobacco chloroplast DNA; psbb: PCR amplified photosystem II chlorophyll a binding 47 kD protein from tobacco chloroplast DNA; psbd: PCR amplified photosystem 11 subunit D2 from tobacco chloroplast DNA. PCR amplified probes comprise the gene coding region, verified by sequencing using the PCR primers prior to labeling.

FIG. 7. Chlorophyll and fluorescence measurements of wild-type tobacco and AtMinD overexpressing tobacco plants.

FIG. 8. Alignment of the Arabidopsis thaliana MinE protein with the MinE protein from other organisms. Syne: Synechocystis sp. (GenBank BAA10661); Guill: Guillardia theta (GenBank AAC35620); Chlorel: Chlorella protothecoides (GenBank CAB42593); Ecoli: Escherichia coli 0157:H7 (GenBank BAB35091); Neiss: Neisseria meningitidis Z2491 (GenBank CAB83414); Pseudo: Pseudomonas aeruginosa (GenBank AAG06633). Symbols in the Arabidopsis MinE1 gene: the downward-pointing arrow indicates a putative chloroplast transit-peptide-processing site, and the inverted black triangle shows the location of the intron.

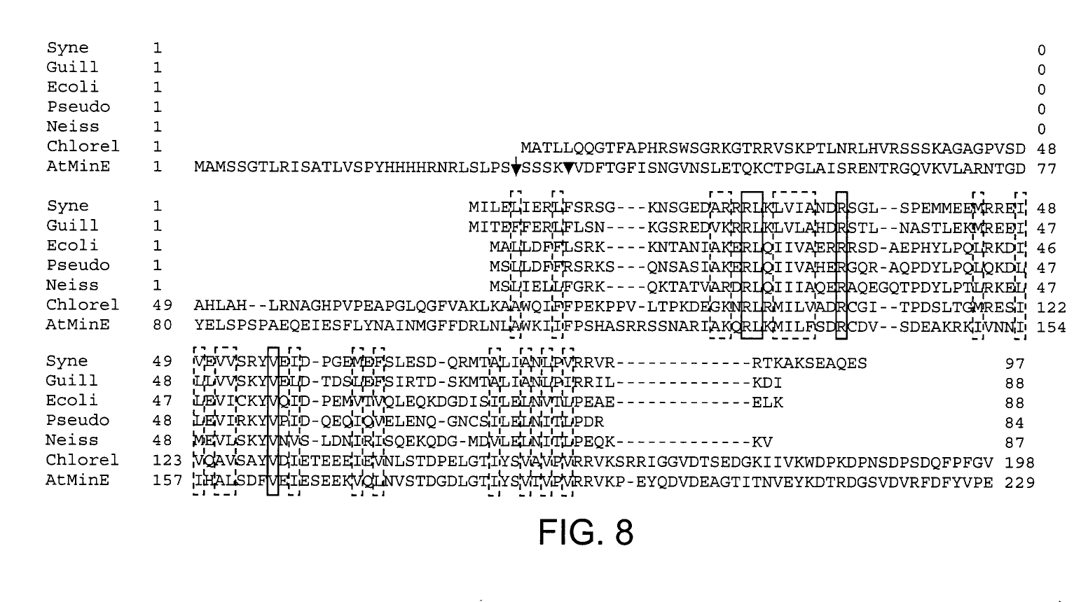

FIG. 9. RNA blots of AtMinE1 expression in Arabidopsis and tobacco. An ethidium bromide-stained gel for each blot is shown below each blot. A) Wild-type AtMinE1 expression in Arabidopsis tissues. FL Flower, RL rosette leaves, CL cauline leaves, AB axillary bud, ST stem. B) and C) AtMinE1 expression in overexpressing lines of transgenic Arabidopsis B) and tobacco C). Wt Wild-type Arabidopsis, ecotype Columbia, in B) and tobacco cultivar KY160 in C) were the non-transgenic controls.

FIG. 10. Confocal images of transgenic Arabidopsis and tobacco expressing the AtMinE1 gene in the sense orientation. A) Wild-type Arabidopsis; B) AtMinE1 Arabidopsis line 3; C) AtMinE1 Arabidopsis line 5; D) Wild-type tobacco; E) AtMinE1 tobacco line 1; F) AtMinE1 tobacco line 3. Each plate is a composite of 16 or 32 frames using the extended focus from the Leica TCS PowerScan software to give a false 3D image. All images were viewed using a 100× objective lens and a resolution of 512×512 pixels (x-y axis). The z axis was composed of 16 frames equally spaced through 21.0 μm (A), 24.0 μm (B), 30.4 μm (C), 34.8 μm (D), 26.1 μm (E) and 43.5 μm (F) of the abaxial leaf surface. Bars=10 μm.

FIG. 11. Transmission electron micrographs of transgenic Arabidopsis and tobacco cells expressing the AtMinE1 gene in the sense orientation. A) Wild-type Arabidopsis; B) AtMinE1 Arabidopsis line 3; C) AtMinE1 Arabidopsis line 5; D) Wild-type tobacco; E) AtMinE1 tobacco line 1; F) AtMinE1 Arabidopsis line 3 (close up). Bars=2 μm (A-E), 0.5 μm (F).

FIG. 12. Electron micrographs of cells from transgenic Arabidopsis plants expressing the AtMinE1 gene in the sense orientation. A) Wild-type Arabidopsis grown in tissue culture; B), C) antisense AtMinE1 Arabidopsis line. Bars=2 μm (A, B), 0.5 μm (C).

FIG. 13. Confocal images of AtMinE1::GFP fusion protein expression. Transient expression of the AtMinE-GFP construct (A-C) and smGFP (D-F) in tobacco (Nicotiana tabacum) leaves monitored 24-36 hours following particle bombardment. A), D) GFP fluorescence monitored at 580-520 nm. B), E) Chlorophyll fluorescence monitored at >645 nm. C), F) Overlay of the GFP fluorescence and chlorophyll fluorescence. Each plate is composite of 30 images collected and overlayed using the extended focus from the Leica TCS PowerScan software to give a false 3D image. Images were viewed using a 40× objective lens and a resolution of 512×512 pixels (x-y axis). For A)-C) the xy axis was 146 μm and for D)-F) 254 μm. The z axis was composed of 30 frames equally spaced through 50 μm A)-C) and 63 μm D)-F) of the abaxial leaf surface. Bar=10 μm.

### DETAILED DESCRIPTION OF THE INVENTION

The present invention fulfills the above-described and other needs by providing a method for a more efficient chloroplast genome transformation in plants by overexpressing a foreign MinD or MinE gene or a foreign gene which is homologous to the Arabidopsis thaliana MinD or MinE gene. By a MinD or MinE gene homologous to the Arabidopsis thaliana MinD or MinE gene it is intended to mean a gene which is identical in sequence to the MinD or MinE gene of Arabidopsis thaliana, or a gene which encodes a protein which has the same activity as Arabidopsis thaliana MinD or MinD protein, or a gene which is at least about 80% similar to the MinD or MinE gene of Arabidopsis thaliana, preferably at least about 85%, and more preferably at least about 90% or 95% similar to the MinD or MinE gene of Arabidopsis thaliana wherein said gene encodes a protein which has the same functional activity as the Arabidopsis thaliana MinD or MinE protein. By a protein having the same functional activity as the MinD or MinE protein of Arabidopsis thaliana it is meant a protein which when transformed into the nuclear genome of a plant results in the production of fewer and larger chloroplasts in the plant.

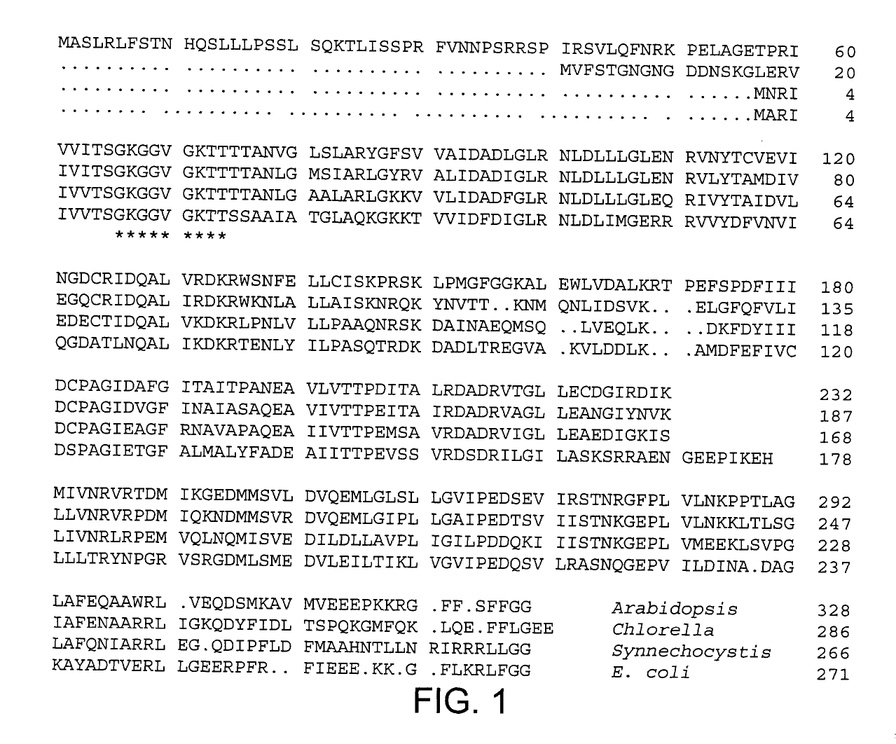

The bacterial MinD gene homologue from the small cruciferous model plant Arabidopsis thaliana (AtMinD) (GenBank Acc. No. AB030278) and the MinE bacterial gene homologue (Protein F23010.25 GenBank Acc. No. AC018364) were identified and isolated by PCR. There is a putative chloroplast targeting sequence present at the amino terminal of the protein expressed by Arabidopsis MinD gene homologue that is not present in bacterial genes (FIG. 1). MinD gene is a normal component in chloroplast division, is expressed in the nucleus and the expressed protein is imported into the chloroplast. Under normal conditions, the MinD protein interacts with genes in the chloroplast to insure that chloroplast division is normal. However, when it is expressed at high levels, MinD gene functions by inhibiting chloroplast division.

Although the examples of the present invention use tobacco plants, any plant which contains chloroplasts can be used in the present invention, including both monocotyledonous and dicotyledonous plants. A number of effective DNA-delivery systems are available for the transfer of exogenous genes into plant genomes and such systems are well known to those skilled in the art. Effective gene transfer into tobacco plant using a vector has been demonstrated.

Tobacco plants were developed which have only one or a few large chloroplasts per cell by expressing the MinD or MinE gene from Arabidopsis thaliana using the CaMV 35S promoter. One of skill in the art would appreciate that MinD and MinE genes from other plant species could also be used to practice the present invention. These chloroplasts were at least 50% fewer or preferably at least 60%, 70%, 80%, 90%, 95% fewer or more preferably at least 98% fewer in number and at least 1× larger or preferably at least 2× larger or more preferably at least 3× larger in size compared to the chloroplasts of wild-type plants. These MinD and MinE overexpressing transgenic tobacco plants appear phenotypically normal in every respect compared to wild-type tobacco plants when grown in the greenhouse. Although expression of a single MinD or MinE gene from Arabidopsis thaliana in tobacco plants using the CaMV 35S promoter results in fewer and larger chloroplasts, nuclear transformation of tobacco plants with multiple copies of the MinD or MinE gene have revealed different results. One possible effect of nuclear transformation with multiple copies of the MinD or MinE gene is that it silences its activity, which results in no change in the morphology of the chloroplast in the transgenic plant. Another possible effect is that it enhances chloroplast division, producing smaller and more numerous chloroplasts in the transgenic plants.

Transmission electron microscopy and laser scanning confocal microscopy of the MinD transgenic plants revealed fewer and abnormally large chloroplasts compared to wild-type tobacco plants which have 10-100 chloroplasts per cell. These large chloroplasts were more conspicuous in mesophyll cells than in the guard cells, possibly due to the differential expression of CaMV 35S promoter in the two cell types. Molecular analysis of these MinD transgenic plants has shown that nuclear and chloroplast gene expression is normal (Dinkins et al, 2001). The large chloroplasts resemble in many respects the single chloroplasts of the algae, Chlamydomonas, that has become the model system for chloroplast transformation.

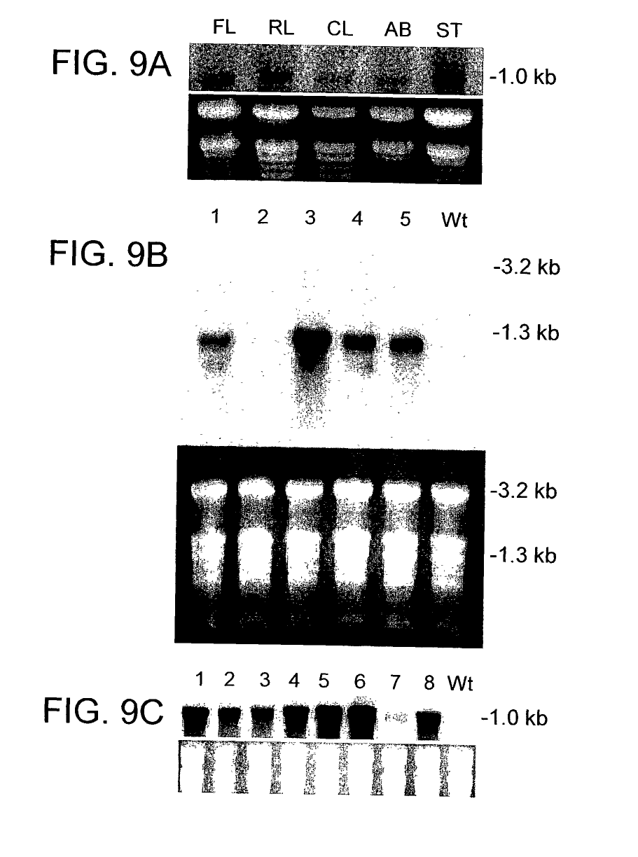

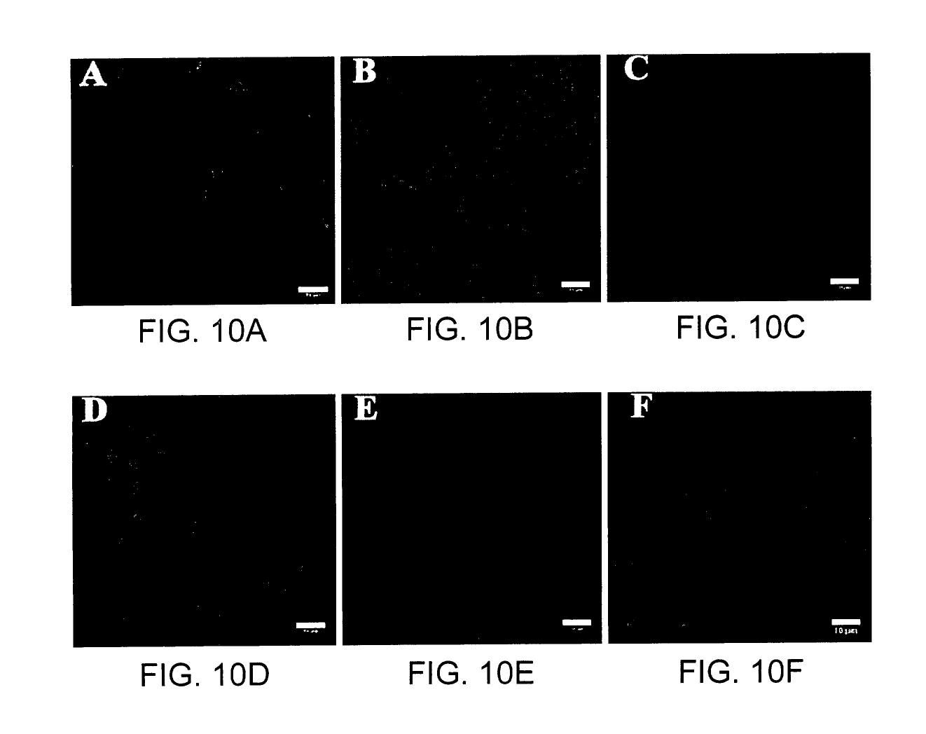

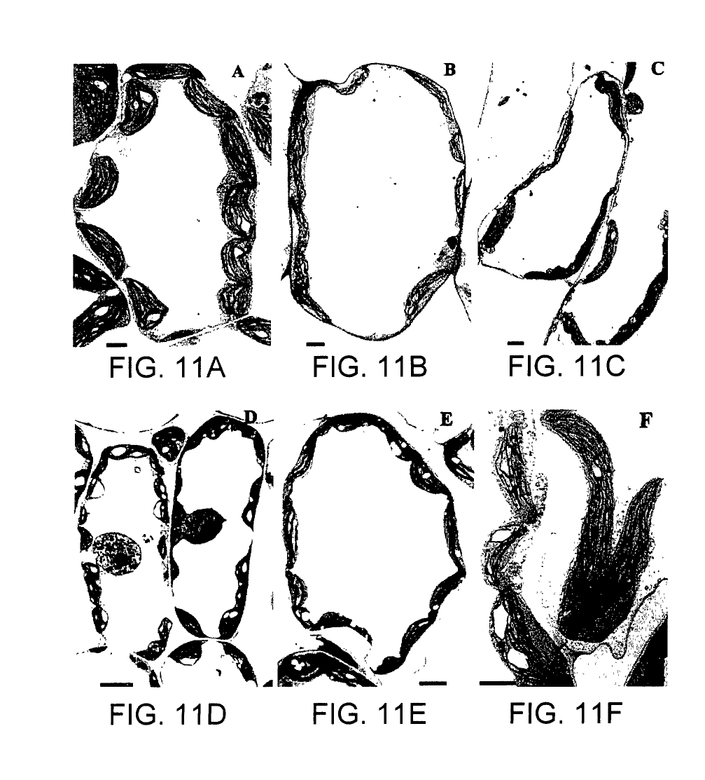

Over-expression of the AtMinE1 gene in Arabidopsis in the sense orientation resulted in a range of chloroplast morphologies as observed using in vivo fluorescence and confocal microscopy (FIG. 10). Many differing shapes of chloroplasts, ranging from normal to very large within the same cell, were evident in transgenic plant cells but not seen in wild-type plants (FIGS. 10B, C). Some of the chloroplasts appeared to be connected by thin strands, almost as if they were in the process of dividing, and the plane of division appeared to be random with regards to orientation. The abnormal morphology and increased size of the chloroplasts were confirmed in electron micrographs of leaf sections (FIG. 11). Chloroplasts of different sizes could be seen, ranging from relatively normal to much larger (FIG. 1F). It was not possible to unequivocally determine actual sizes of chloroplasts in the electron micrographs, as the plane of the section of some of the chloroplasts was probably not longitudinal. The thylakoid membranes appeared normal and were continuous within the large chloroplasts. Arabidopsis line sense #3 had the highest expression of the transgene and most of the chloroplasts in this line appeared larger and were morpholobically abnormal compared to the wild type (FIGS. 9B and 10B). Abnormal chloroplasts were found in the AtMinE1 transgenic lines, including Line 2; however, consistent with the level of transgene expression, Line 2 had only a few cells in which larger abnormal chloroplasts were detected by either confocal or electron microscopy.

To determine the effect of AtMinE1 gene expression in a heterologous system, the AtMinE1 gene was transformed into tobacco. AtMinE1 expression in tobacco plants provides an indication of the function of the AtMinE1 protein in a heterologous system without the problems that may occur due to gene silencing (Depicker and van Montagu 1997; Smyth 1999) since the AtMinE1 gene did not cross-hybridize with a tobacco homologue, even when the blot was overexposed (FIG. 9C). Chloroplast morphology in the AtMinE1 tobacco plants was similar to that of the AtMinE1 transgenic Arabidopsis plants. Both confocal and electron micrographs showed that the cells contained abnormally shaped and variable-sized chloroplasts (FIGS. 10E, F; FIG. 11D, E). Abnormally shaped chloroplasts were observed in the guard cells, with some appearing to be attached or in the process of dividing (FIG. 10F); these chloroplasts were not as large as those observed in the mesophyll cells (FIGS. 10E, F).

No additional visible phenotypic abnormalities were observed with any of the AtMinE1-overexpressing Arabidopsis or tobacco lines. Photosynthetic electron transport, as measured by fluorescence-induction kinetics, and chlorophyll content were measured in the tobacco lines and were not found to be significantly different from the wild-type tobacco.

The plants analyzed in this study were grown under greenhouse conditions, and may have a more striking phenotype than would be the case under normal field conditions. It would be expected that the extreme disruption of the chloroplast morphology would confer some developmental or growth disadvantage. On the other hand, Pyke et al. (Plant Physiol. 106:1169-1177 (1994)) have demonstrated that the Arabidopsis mutant, arc6, that has only two chloroplasts per cell, appears to be phenotypically normal except for some curling of the rosette leaves. A few of the AtMinD overexpressing tobacco plants exhibited some chlorophyll deficiency and reduced fertility. However, it is not uncommon to recover occasional transgenic KY160 tobacco plants that are lighter in color and have reduced fertility presumably due to the tissue culture process.

To assess the MinD transgenic tobacco lines, molecular analysis was done to compare the MinD transgenic lines and wild-type as to the progress towards the transgenic homoplastic condition. This is done by performing a Southern blot on the developing plantlets in culture. Cells that contain a mixed population of chloroplasts, or chloroplasts that have not reached a homoplastic state, can be identified by having two bands on a Southern blot instead of the expected one. In addition, the homoplastic transgenic plants will show the inserted DNA, such as the spectinomycin gene, whereas the wild-type will not.

Prior art chloroplast transformation protocols, while published and available, are not on par with the nuclear transformation of Agrobacterium tumefaciens and particle bombardment procedures. Chloroplast transformation continues to be a low efficiency and time consuming process due to the requirements for continuing sub-culturing to obtain homoplastic transgenic plants. Using the MinD overexpressing lines, and other lines encompassed by the present invention, an increase in the efficiency of chloroplast transformation can be obtained.

Particle bombardment, in particular, has been used successfully to obtain fertile transgenic chloroplast plants in tobacco, potato and Arabidopsis thaliana. Since the introduced DNA must integrate into the chloroplast chromosome through homologous recombination, the problems associated with illegitimate recombination in the nuclear genome are not found. However, delivery of gold particles coated with the DNA of interest into chloroplasts is dependent on gold particle size and pressure used. The use of the MinD overexpressing lines, and other lines encompassed by the present invention, which have the large chloroplasts, increases transformation efficiency.

An exemplary vector used by the inventors for chloroplast transformation contained the spectinomycin resistance gene and the Bacillus thuringiensis (Bt) protein. While the spectinomycin resistance gene may be of interest, the use of the Bt gene in tobacco has already been demonstrated (McBride et al., Bio/Tech. 13:362-365 (1995)). One of skill in the art would know of other suitable genes which can be used in the vector of the present invention. For example, genes involved in the polybutyric acid pathway; genes expressing the Δ-endotoxin gene; and antibody vaccine genes for humans and animals can be used in the vector of the present invention. Examples of promoter and terminator sequences which can be used in the vector of the present invention should be bacterial or derived from the chloroplast. A strong promoter for expression in chloroplasts include psbA or psbD/C under light conditions, or the rbcL and 16S ribosomal RNA promoter for ubiquitous expression.

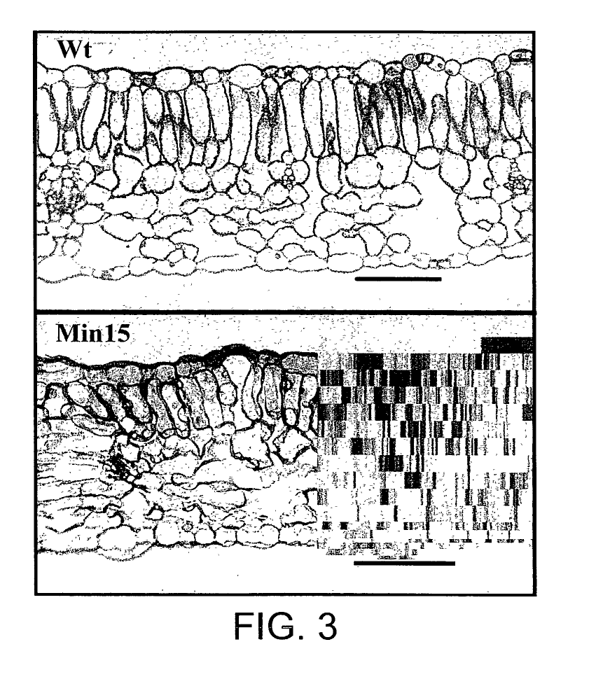

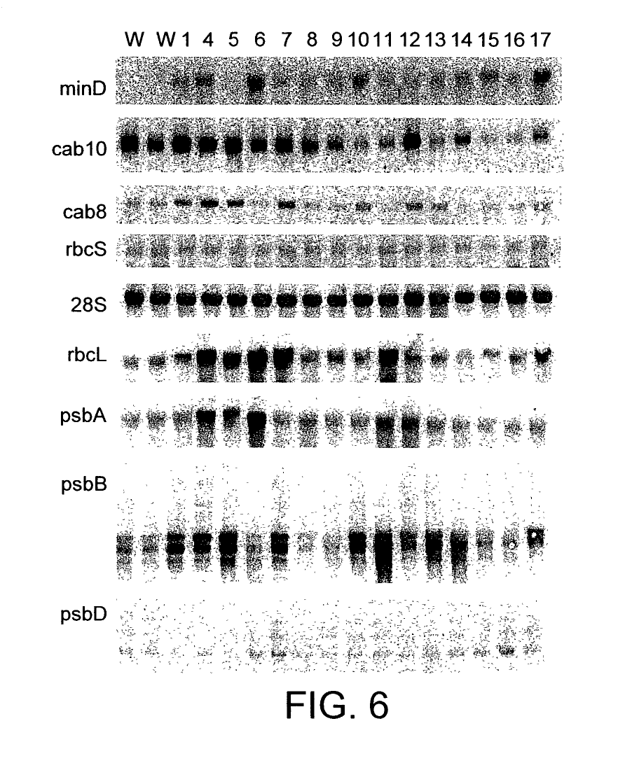

In the present invention there was some variation observed in the number of chloroplasts per cell in some of the transgenic tobacco lines. Most of the lines had 1-2 or a few chloroplasts per cell, while other lines had distinctly larger chloroplasts, with 5-8 chloroplasts per cell. Some of this variability may be due to the AtMinD or AtMinE transgene expression level in these lines. A correlation was observed between the AtMinD RNA level and number of abnormal chloroplasts in the overexpressing plants. This was especially evident in the guard cells. The mesophyll cells, for the most part, all contained abnormally large chloroplasts in all of the overexpressing lines, except for line #5 which contained both normal and abnormal chloroplasts (FIG. 3B). This line also had the lowest level of AtMinD expression (FIG. 6). Moderate expression of AtMinD resulted in plants with abnormal mesophyll cell chloroplasts and normal guard cell chloroplasts, or plants with abnormal mesophyll cell chloroplasts and both normal and abnormal chloroplasts in the guard cells. In the high expressing lines, all cells, guard and mesophyll, had large abnormal chloroplasts.

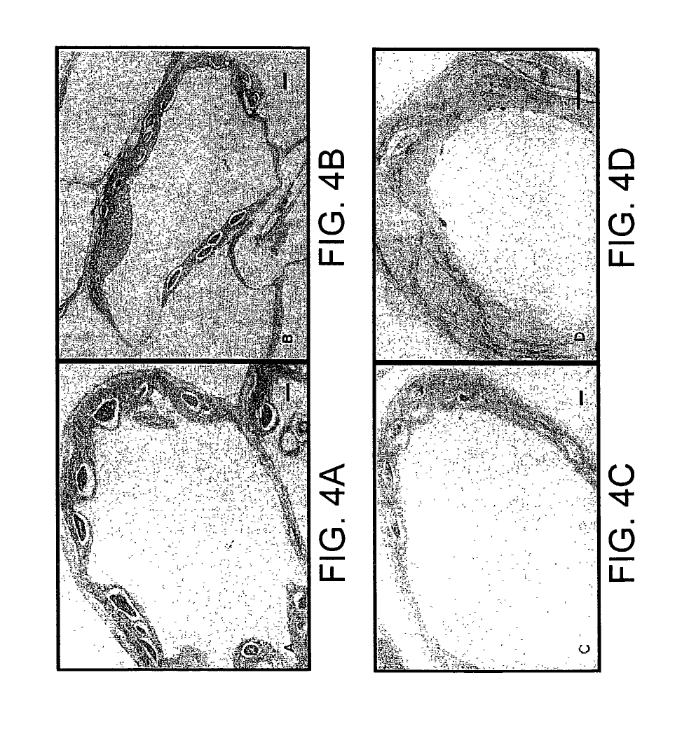

As shown in FIG. 4D, electron microscopy revealed that the thylakoid membranes were normal throughout the chloroplast in the AtMinD transgenic tobacco plants, and that they were contiguous. Confocal microscopic analysis of the live tissue using fluorescence to view chloroplast structure corroborated the normal functioning of the AtMinD tobacco chloroplasts. None of the plants displayed abnormally high fluorescence that is usually observed when photosynthetic electron transport is abnormal or disrupted (Miles, Maydica 39:35-45 (1994)). In the tobacco AtMinD transgenic lines, the fluorescence appears as sheets layered on top of each other throughout the length of entire chloroplast. This type of fluorescence is more commonly observed in the agranule bundle sheath chloroplasts of C4 plants, such as maize or sorghum (Mehta et al., Aus. J. Pl. Physiol. 26:709-716 (1999)). While the results obtained from the confocal images of the AtMinD overexpressing lines are not completely understood at this time, the confocal images confirm that the chloroplasts in these lines are not dividing normally, and that there is continuity of the thylakoid membranes throughout the chloroplast. Some of the large chloroplasts extend the entire circumference of the cell.

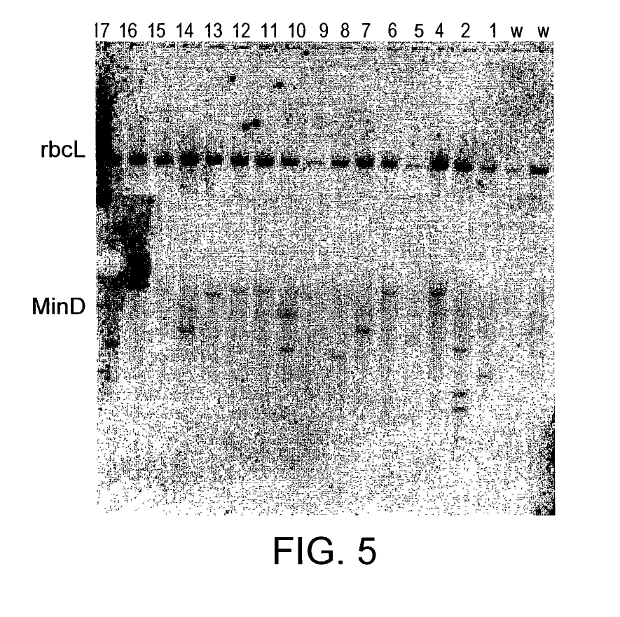

In spite of the differences in chloroplast morphology, there did not appear to be any consistent differences in chloroplast gene expression or nuclear gene expression of chloroplast directed proteins in the AtMinD overexpressing plants. Although differences were observed, none of the differences could be attributed to higher or lower AtMinD RNA levels in the transgenic lines. It has been previously shown that much of the regulation of chloroplast genes occurs post-transcriptionally (Deng and Gruissem, Cell 49:379-387 (1987); Gruissem et al., Trends Genet. 4:258-263 (1992)). No differences were seen in the concentration of chloroplast DNA molecules as measured by the relative level of the rbcL gene (FIG. 5). These results suggest that while chloroplast division was inhibited in the AtMinD transgenic plants, chloroplast DNA replication was normal.

The present invention further provides a method to return the plants that are chloroplast transgenics to normal chloroplast morphology to avoid possible future adverse effect. This can be easily achieved, by the present invention, through segregating out the nuclear overexpressed MinD or MinE transgene by crossing with normal tobacco plants and selecting plants that are chloroplast transgenics (i.e., they have the desired characteristic produced by the exogenous gene expressed in the chloroplast genome), with a normal size and number of chloroplasts, but not nuclear transgenics (which have a few large chloroplasts).

All of the articles and patents cited herein are incorporated by reference in their entirety.

The following examples are presented in order to more fully illustrate the preferred embodiments of the invention. They should in no way be construed, however, as limiting the broad scope of the invention.

### EXAMPLE 1

In an effort to determine a role for the MinD gene, an Arabidopsis thaliana putative MinD homologue (AtMinD) was isolated. Oligonucleotide primers were synthesized based on the sequence for the bacterial MinD gene, MZF18.5, on bacterial chromosome 5 [5′ Forward: TCTCGAGAATGGCGTCTCTGAGATTGTTC; 3′ Reverse: TTCTAGATTTGCCATTTAGCCGCCAAAG]. The primers were synthesized to include the ATG start site and the TAA stop codon (underlined above) and to include an XhoI and XbaI restriction endonuclease site at the 5′ and 3′ ends, respectively. Total DNA from the Arabidopsis thaliana strain, Columbia, was used for amplification in a standard PCR reaction buffer [20 mM Tris-HCl (pH 8.0), 2.0 mM MgCl₂, 0.25 mM of each dNTP, 100 ng of each primer and 2 units of Taq DNA polymerase (Gibco/BRL, Rockville Md.)]. The DNA fragment was cloned into pGEM T-(Promega, Madison, Wis.) and verified by sequencing. The XhoI-XbaI MinD gene fragment was excised from pGEM and cloned into the SalI and XbaI sites of the pKYLX71 binary vector containing the caMV 35S² promoter for constitutive overexpression (Maiti et al., Proc. Natl. Acad. Sci. USA 90:6110-6114 (1993)). All E. coli manipulations were carried out in the strain TB1. The pKYLX71:AtMinD recombinant plasmid was mobilized into Agrobacterium tumefaciens C58C1:pGV3850 by tri-parental mating (Schardl et al., Gene 61:1-11 (1987)). Agrobacterium tumefaciens mediated transformation of tobacco plants was performed using the protocols described in Schardl et al., (1987).

Several transformation experiments were conducted and a large number of fertile transgenic tobacco plants were obtained (˜100). Initial experiments were done with T₀ plants that were transferred to the greenhouse. At the whole plant level, all of the transgenic tobacco lines overexpressing the AtMinD gene appeared essentially normal under greenhouse conditions. Although several of the AtMinD plants were slightly pale and grew slower than the normal KY160 parental line, there were no major phenotypic differences, such as plant size or fertility, when compared to the wild-type KY160 tobacco cultivar.

### EXAMPLE 2

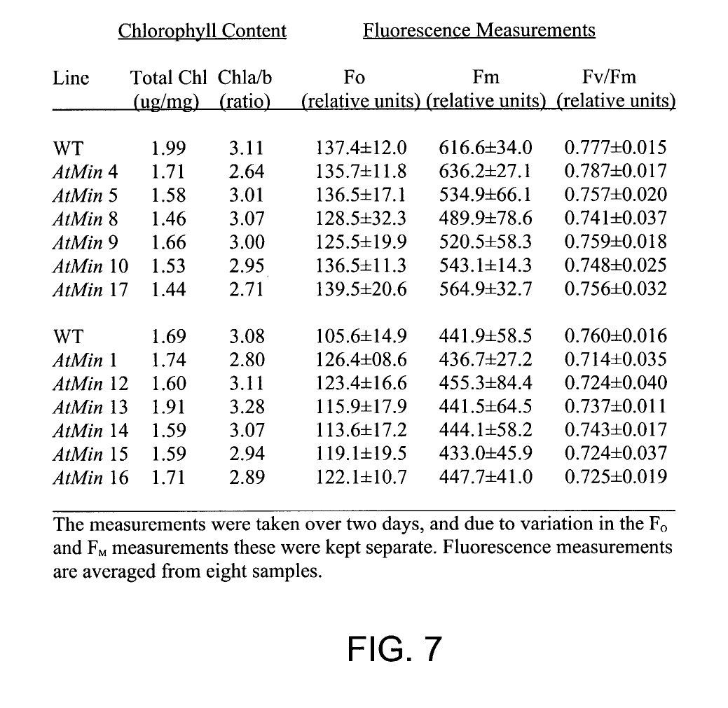

In addition to visual ratings, further analysis was done on the first fifteen lines that were established in the greenhouse. Chlorophyll analysis suggested that several of the lines, namely AtMinD lines 8, 9 and 10, had less total chlorophyll, and alone with line 17, the chlorophyll a to chlorophyll b ratio was slightly reduced (FIG. 7).

Photosynthetic electron transport rates measured by fluorescence kinetics were slightly lower in some of the lines, but overall F₀, F_{M} and F_{V}/F_{M} were not significantly different from the wild-type KY160 in most of the lines (FIG. 7). Only three lines (8, 9 and 10) showed a significant reduction in the maximal fluorescence (F_{M}), a lower but not significant reduction in initial fluorescence (F₀) as well as overall kinetics (F_{V}/F_{M}). This suggests that while there may be less functional photosystem II centers, photosynthetic electron transport is not limiting.

### EXAMPLE 3

Chloroplast morphology and ultrastructure were observed using microscopic analysis. Leaf samples from greenhouse grown AtMinD transgenic and wild-type plants were trimmed into 1×1 mm squares, then prefixed in 3% glutaraldehyde in 0.1 M cacodylate buffer, pH 7.2, for 2 hr at room temperature. Samples were washed twice with 0.1 M cacodylate buffer, pH 7.2, then postfixed in 1% osmium tetroxide in the same buffer for 2 hr at 4° C. After rinsing in deionized water, the samples were stained overnight in aqueous saturated uranyl acetate at 4° C. Samples were rinsed in deionized water, then dehydrated though an ethanol series, followed by treatment with 100% acetone and infiltrated and embedded in Spurr's resin. Polymerization took place overnight at 70° C.

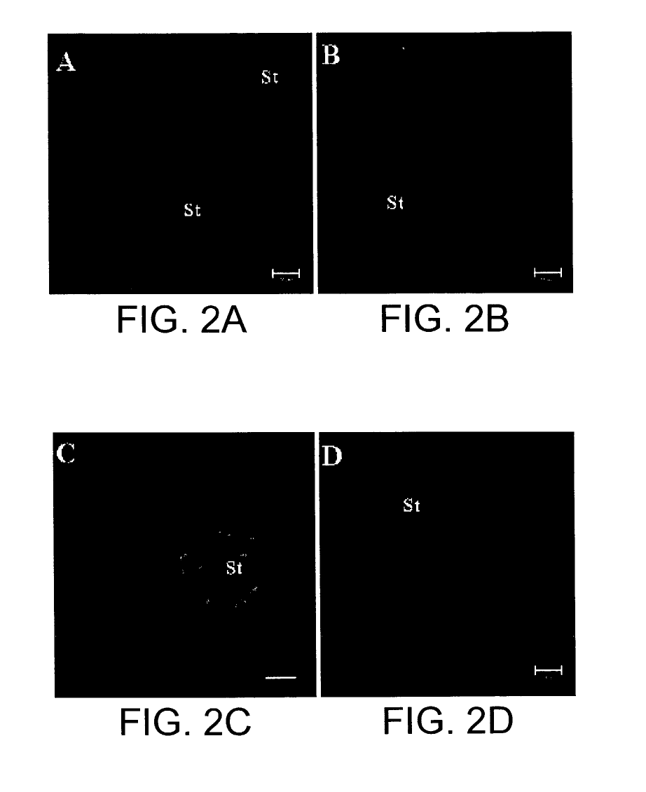

Thin sections (2 μM) of the leaf samples were stained with 1% Toluidine Blue and examined under a Zeiss optical microscopy. Microscopic analysis revealed that the chloroplasts in the over-expressing lines were abnormal (FIG. 2). Under light microscopy, the mesophyll cells appeared to have a single large continuous chloroplast. The folds within some of these cells make it difficult to discern whether the chloroplast structure is drastically different from that of wild-type chloroplasts.

To confirm the continuity of the chloroplasts, chloroplasts in single cells were optically scanned using a confocal laser scanning microscope by visualizing chloroplast fluorescence (FIG. 3). For Confocal Scanning Laser Microscopy-(CSLM), leaf samples from greenhouse grown AtMinD transgenic and wild-type plants were cut from fully developed mature leaves, and all the major veins removed. The leaf samples were directly mounted on a microscope slide in a buffer consisting of 90% glycerol and 10% 0.05 M Sodium Phosphate (pH 6.0) to aid in sample immobilization. Confocal microscopy was performed with a Leica TCS (Leica Microsystems Heidelberg GmbH, Heidelberg, Germany) using both the FITC and TRITC filters. The objective lens was a Leica 100× oil immersion with a numerical aperture of 1.2 and a working distance of 100-300 um. An argon laser was used to excite the chlorophyll molecules in the leaf at a wavelength of 488/20. A sequence of 16 x-y optical slices was collected through the abaxial leaf orientation, with distances through the z axis (25-40 um for whole leaves and 8-16 um for single cells) for each sample as indicated in the figure legend.

The software used for imaging was the Leica TCS PowerScan™ software run on a Windows NT operating system. The images were collected using the medium speed with a 512×512 resolution for 4 or 8 passes through the 16 sections. Fluorescence intensity was digitally coded using 256 levels of gray, with 0 representing the lowest intensity (black) and 255 the highest (white). The extended focus on the software program was used to view the apparent depth giving a false 3D image and included both filters.

Images were observed through both the adaxial and abaxial orientations and the chloroplast morphology was similar in both the palisade and spongy mesophyll cells. Due to the compactness and shape of the palisade mesophyll cells, resolving distinct chloroplasts was easier scanning through the spongy mesophyll, thus the abaxial leaf orientation was subsequently used. In addition, the abaxial surface has fewer trichomes, which in tobacco contain autofluorescing compounds that make imaging more difficult at the leaf surface. Visualizing the fluorescence with the confocal microscope, three distinct chloroplast phenotypes were observed. First, in the mesophyll cells of most of the AtMinD overexpressing plants, we observed that the cells possessed a few (1-4) very large chloroplasts that appeared to occupy the entire cell. In plants displaying an intermediate phenotype, the cells contained 5-10 large chloroplasts throughout the cell. Plants of the third phenotype had some cells with abnormally large chloroplasts and other cells with normal chloroplast number and size. The extent of the abnormality in chloroplast size correlated to the level of AtMinD transgene expression in these lines (see Example 4).

Another interesting observation in chloroplast morphology in most of the AtMinD overexpressing lines was noted in the guard cells. Guard cells appear to be less affected than the mesophyll cells in the alteration in the number of abnormal chloroplasts. In most of the AtMinD transgenic lines, the chloroplasts in the guard cells appeared to be normal in size and number (FIGS. 3B-C). Some of the transgenic lines contained guard cells with both normal and abnormal chloroplasts. Only in those lines with high AtMinD expression were all the chloroplasts in all the guard cells abnormal (FIG. 3D).

In order to determine whether the thylakoid membrane was normal in the AtMinD overexpressing lines, Transmission Electron Microscopy (TEM) was performed (FIG. 4). For TEM leaf samples from greenhouse grown AtMinD transgenic and wild-type plants were prepared as described for Light microscopy above. Ultrathin sections (approximately 0.07 μM) were cut with a glass knife on a SORVALL MT-2 ultra-microtome, recovered on a grid and stained with lead citrate. Stained sections were viewed in a HITACHI H-600 Transmission Electron Microscope.

The continuity of the thylakoid membranes in the chloroplast in the AtMinD overexpressing lines could be readily observed throughout the entire chloroplast with the help of TEM (FIG. 4D). The thylakoid membranes overlap continually throughout the entire chloroplast. The size of the chloroplast does not affect the thylakoid membrane structure, as it appears normal with respect to the presence of both granna and stromal lammelae. While an extensive survey was not conducted, the few mitochondria that were seen using the electron microscope were normal irrespective of the AtMinD expression level.

### EXAMPLE 4

Southern analysis was performed to determine number of inserted copies of AtMinD transgenes (FIG. 5). Putative transgenic plants were screened for the presence of the transgenes by Southern blot hybridization according to method taught by Sambrook et al. (Molecular Cloning: A Laboratory Manual, 2nd Ed., Cold Spring Harbor Laboratory Press (1989)). Tobacco genomic DNA was isolated by homogenizing 100 mg leaf tissue in a 1.5 ml microfuge tube containing 500 μl of extraction buffer (100 mM Tris-HCl pH 8.0, 20 mM EDTA, 0.5 M NaCl and 0.5% w/v SDS, 0.5% v/v β-mercaptoethanol). The ground extract was treated with 500 μl of a phenol:chloroform:isoamyl alcohol mixture (25:24:1) and centrifuged at 13,000 rpm for 5 min. The aqueous phase was collected, mixed with 1 μl of 10 mg/ml RNase A and incubated at room temperature for 20 min. The samples were re-extracted with an equal volume of phenol:chloroform:isoamyl alcohol (25:24:1), followed by two re-extractions chloroform:isoamyl alcohol (24:1). DNA was precipitated by adding 2.5 volumes of ethanol and spooled out. The spooled DNA was washed in 70% ethanol, dried and resuspended in 100 μl sterile water.

Five μg of DNA from each plant was digested overnight with XhoI, separated on a 0.8% agarose gel and blotted onto ‘Zetaprobe’ membrane (BioRad Laboratories, Hercules, Calif.). XhoI does not cut within the T-DNA and thus generates bands that are indicative of the number of AtMinD inserts into the genome. Hybridizations were carried out with random primed α³²P-dCTP labeled probes using the Prime-It© II Random Primer Labeling Kit (Stratagene, La Jolla, Calif.) in formamide: White Rain® moisturizing formula hair shampoo (The Gillette Company, Boston, Mass.): deionized water, (5:4:1) solution overnight at 42° C. The membrane was then washed three times at room temperature in 0.1× SSC and 0.1% SDS, and then at 42° C. for 1 hour, and was exposed in a phosphorimager cassette (Molecular Dynamics, Inc; Sunnyvale, Calif.). Additional washings at higher temperatures were carried out as necessary.

Most of the AtMinD transgenic tobacco lines had a single copy of the T-DNA (lines AtMinD 1, 4, 6, 7, 8, 9, 11, 12, 13, 14, 15, 16 and 17), with one line containing two copies (AtMinD transgenic line 10) and two lines with multiple copies (AtMinD transgenic lines 2 and 5) (FIG. 5). The moderate stringency wash showed that the AtMinD gene does not cross hybridize with a tobacco MinD homologue. A Southern blot was also done using the rbcL gene as a probe (FIG. 5). No differences other than DNA loading were observed for the rbcL gene. This suggests that while chloroplast division is repressed in the transgenic plants, there is basically no effect on chloroplast DNA replication.

### EXAMPLE 5

Chloroplast expression of the AtMinD gene was determined using RNA blots.

Total RNA was isolated from AtMinD tobacco greenhouse grown plants (˜100 mg) using 1 ml of Trizol reagent (Gibco/BRL, Rockville, Md.). 10 μg of total RNA was separated on 1.0% formaldehyde containing agarose gels and transferred onto a ‘Zetaprobe’ membrane (BioRad Laboratories, Hercules, Calif.). Hybridization and washing were done as described in Southern Analysis. No cross hybridization was observed with wild-type tobacco RNA.

The number of transgene insertions did not appear to be a factor in the expression of the AtMinD transgene in the tobacco (FIG. 6). However, a strong correlation between AtMinD expression and the number and size of the abnormal chloroplasts was observed. Line AtMinD 5 had the lowest transgene expression, and most of the cells contained normal chloroplasts (FIG. 3B). Some of the cells of this line contained abnormal chloroplasts, but these were the exception rather than the rule. Transgenic AtMinD lines with an intermediate level of expression (lines 1, 4, 7, 8, 9, 11, 12, 13, 14, 15 and 16) had abnormal chloroplasts in the mesophyll cells, but the chloroplasts in the guard cells were either normal, or in some cases, both normal and abnormal. High AtMinD expressing lines (lines 6, 10 and 17) had no normal chloroplasts in either the mesophyll or guard cells (FIG. 3D).

While variations were observed in the steady state RNA levels of several nuclear encoded, chloroplast-directed gene products (cab8, cab10 and rbcS) and chloroplast operons (atpA, atpB/E, psaB, psbA, psbB, psbD, petA, and rbcL), there were no apparent or consistent differences seen due to the overexpression of the AtMinD transgene in these plants (FIG. 6). The transcripts that displayed the most differences between the AtMinD tobacco lines were rbcL, psbA, and psbB. Over a two-fold difference was observed with the rbcL gene, with lines 4, 6, 7 and 11 displaying the highest expression levels. Line 6 had high AtHinD expression, but lines 4, 7 and 11 were only moderate overexpressors. Line 17, another high AtMinD expressor, had a moderately high level of rbcL transcript, however, in another high AtMinD expressing line (#10), the rbcL transcript level was low. The psbA transcript was high in tobacco lines AtMinD 6 and 4; both of these lines exhibited high rbcL expression. However, lines 7 and 11 both exhibited low psbA expression. AtMinD tobacco line 7 had high psbB transcript level as did line 11. Line AtMinD 6 also exhibited low psbb expression. AtMinD line 5 also possessed one of the highest levels of psbB transcripts, yet had the lowest AtMinD expression of any of the lines evaluated. Thus, while a significant variation was found in the levels of the different chloroplast transcripts, there was no discernable pattern in transcript level with respect to MinD overexpression.

### EXAMPLE 6

Oligonucleotide primers were synthesized based on the sequence found on bacterial artificial chromosomes (BACs) F23O10.25 and F10D13.22 on chromosome 1. The Arabidopsis MinE1 gene was isolated by reverse transcription (RT)-PCR from RNA isolated from leaf tissues of Arabidopsis thaliana (L.) Heynh., accession line Columbia. For cloning into the KYLX71:35S² binary vector (Maiti et al., Proc. Natl. Acad. Sci USA 90:6110-6114 (1993)), primers (5′ forward: 5′-AGT TTC TCG GTA ATG GCG ATG T-3′; 3′ back: 5′-GAC TGT GCC TTT TCA TCA CTC T-3′) were synthesized to include the ATG start site and TAG stop codon (shown in bold italics) with an addition of an XhoI and XbaI restriction endonuclease site at the 5′ and 3′ end, respectively, for the sense primers, and reversed for the antisense primers. For green fluorescent protein (GFP) fusion protein the same 5′ primer above was used with a 3′ primer (5′-TTG AGC TCA CCT CCA ACA TTA AAA TCG AAC CTG-3′) that deleted the stop codon and contained an SstI endonuclease site immediately following to provide an in-frame sequence with the entire GFP protein. The GFP gene was isolated by PCR from a plasmid carrying mgfp5 (Siemering et al 1996); primers (5′ forward: 5′-TTG AGC TCA TGA GTA AAG GAG AAG AAC T-3′ and 3′ back: 5′-TTC TAG ATT ATT TGT ATA GTT CAT CCA TG-3′) were designed to have SstI and XbaI restriction-endonuclease recognition sites.

Total RNA from the A. thaliana ecotype Columbia was isolated using the Trizol method (Gibco/BRL) and first-strand synthesis was done using the First Strand Synthesis Kit (Stratagene). Following the first-strand synthesis, 1 μl of cDNA was used for amplification in a standard PCR reaction buffer (20 mM Tris-HCl (pH 8.0), 2.0 mM MgCl₂, 0.25 mM of each dNTP, 100 ng of each primer and 2 units of Taq DNA polymerase (Gibco/BRL)). The DNA fragment was cloned into pGEM T-vector (Promega) and verified by sequencing. The XhoI-XbaI AtminE1 gene fragment was excised from pGEM and cloned into the XhoI/XbaI sites of the pKYLX71:35S² vector (Maiti et al. 1993). All plasmid manipulations were carried out in the E. coli TB1 strain. The pKYLX71:AtMinE1 recombinant plasmid was mobilized into Agrobacterium tumefaciens C58C1:pGV3850 by tri-parental mating (Schardl et al, Gene 61:1-11 (1987)).

### EXAMPLE 7

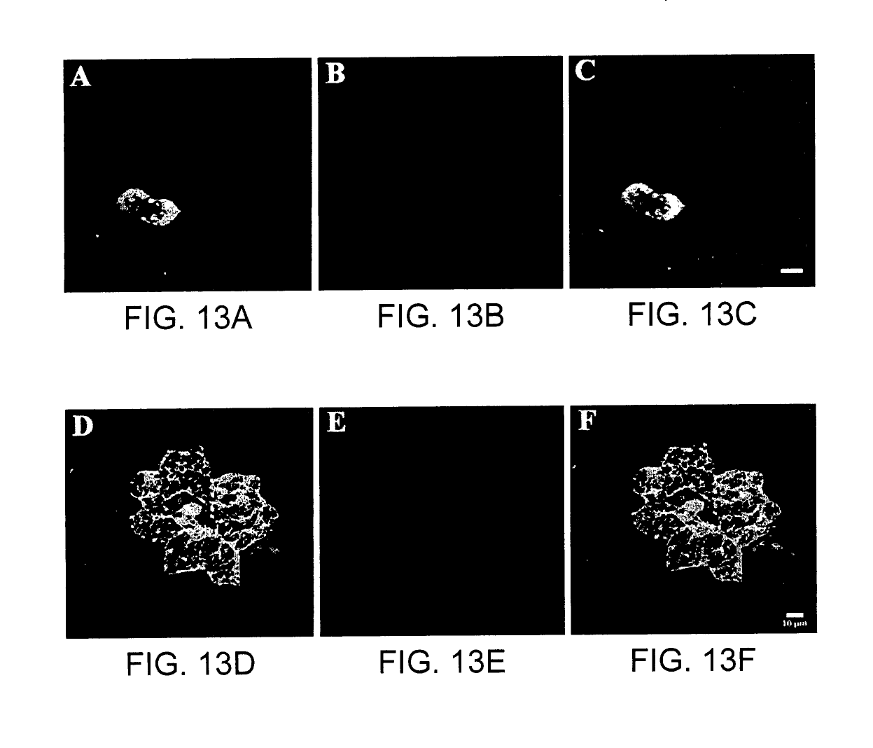

In order to determine if the AtMinE1 protein is targeted to the chloroplast, an AtMinE1::GFP fusion protein was constructed. The AtMinE1 (without a stop codon) XhoI/SstI fragment was cloned into the XhoI/SstI site in pKYLX80, a modified Bluescript vector containing the cauliflower mosaic virus (CaMV)35S² promoter, multiple cloning site and rbcS 3′ terminator from the pKYLX71 vector. The GFP SstI/XbaI fragment was then cloned into the SstI/XbaI sites in the pKYLX80 vector to give an in-frame fusion protein. The plasmid was introduced by particle bombardment (PDS1000; BioRad, Hercules, Calif., USA) into tobacco leaf explants. GFP expression was monitored 24 hours after bombardment. As shown in FIG. 13, the fluorescence from the AtMinE1::GFP fusion protein was observed specifically in the chloroplasts (FIG. 13A) whereas the soluble modified GFP alone was distributed throughout the cell and not associated with the chloroplasts (FIG. 13D). These results demonstrated that the nuclear-encoded AtMinE1::GFP fusion protein was targeted to the chloroplasts.

### EXAMPLE 8

Nicotiana tabacum L. cv. KY160 (University of Kentucky Tobacco Breeding Program), N. tabacum germplasm Petit Havana (seeds from A. G. Hung, University of Kentucky) and Arabidopsis thaliana (L.) Heynh. ecotype Columbia (seeds from D. W. Meinke, Oklahoma State University) were used for plant transformation experiments. Tobacco transformation protocols and media were as in Schardl et al (1987). Initial experiments were done with T₀ plants that were transferred to the greenhouse. Self-fertilized seeds from individual T₀ plants were harvested and 10 T₁ plants were assayed. A. thaliana was transformed using the whole-plant-dip method (Clough and Bent, Plant J. 16:735-743 (1998)).

### EXAMPLE 9

Leaf samples form greenhouse grown_AtMinD1 transgenic and wild-type plants were trimmed into 1 mm×1 mm squares, then pre-fixed in 3.5% glutaraldehyde in 0.1 M Sorenson's buffer (pH 6.8) for 2 hours at room temperature. Samples were washed three times with 0.1 M Sorenson's buffer (pH 6.8), and then post-fixed in 1% osmium tetroxide in the same buffer for 2 hours at room temperature. Samples were rinsed in deionized water, dehydrated through an ethanol series, treated with propylene oxide, and infiltrated and embedded in Spurr's resin. Polymerization took place overnight at 70° C. Ultrathin sections (approx. 0.07 μm) were cut with a diamond knife on a Reichert Ultracult E ultra-microtome, recovered on a grid, and stained with lead citrate. Stained sections were viewed in a Hitachi H-7000 transmission electron microscope at 75 kV.

### EXAMPLE 10

Leaf samples from greenhouse-grown AtMinE1 transgenic and wild-type plants were cut from fully developed mature leaves, and all the major veins removed. The leaf samples were directly mounted on a microscope slide in a buffer consisting of 90% glycerol and 10% 0.05 M sodium phosphate (pH 6.0) to aid in sample immobilization.

Confocal microscopy was performed with a Leica TCS (Leica Microsystems Heidelberg, Germany) microscope. The objective lens was a Leica 100× oil immersion with a numerical aperture of 1.2 and a working distance of 100-300 μm. An argon laser was used to excite the chlorophyll molecules in the leaf at a wavelength of 458±20 nm and 488±20 nm, and two sets of filters were used to collect the data, one at 520-580 nm to visualize GFP fluorescence and the other to pass all light greater than 620 nm to monitor chlorophyll fluorescence. Equally spaced optical slices were collected through the abaxial leaf orientation, with the total distance through the leaf (z axis) for each sample indicated in the figure legend.

The Leica TCS PowerScan software, run on a Windows NT operating system, was used for imaging. The images were collected using the medium speed setting with 512×512 pixel resolution with 4 passes through each of the 16-32 equally spaced sections through the leaf. Fluorescence intensity was digitally coded using 256 levels of gray, with 0 representing the lowest intensity (black) and 255 the highest intensity (white). The three-dimensional (3D) image was created using the extended focus on the software program that included an overlay of all the images collected providing an apparent 3D image.

### EXAMPLE 11

Using the Trizol method (Gibco/BRL) total RNA was isolated at the same time of day from fully expanded leaves of greenhouse-grown plants of AtMinE1 tobacco or Arabidopsis. For analysis of gene expression, RNA from different Arabidopsis tissues, 100-mg samples of flowers, stem, cauline leaves, rosette leaves and siliques, was isolated from wild-type greenhouse-grown Arabidopsis plants by the Trizol method. Ten μg of total RNA was separated on 1.0% formaldehyde-containing agarose gels and transferred onto a Zetaprobe membrane (BioRad Laboratories). Hybridization and washing were done essentially as described in Sambrook et al, Molecular Cloning: A Laboratory Manual, 2nd Edn., Cold Spring Harbor Laboratory Press, Cold Spring Harbor N.Y. (1989). Random primed α-[³²P]dCTP-labelled probes were prepared using the Prime-It II Random Primer Labeling Kit (Stratagene). Hybridizations were carried out in 50% formamide hybridization buffer solution (0.12 M Na₂HPO₄, 0.25 M NaCl, 7% v/v SDS 1 mM EDTA) overnight at 42° C. The membrane was then washed three times at room temperature in 0.2× SSC (1× SSC is 0.15 M NaCl and 0.015 M sodium citrate) and 0.1% SDS and was exposed in a phosphorimager cassette (Molecular Dynamics). Additional washings at higher temperatures were carried out as necessary.

### EXAMPLE 12

For stable transformation, the AtMinE1 gene was cloned into the Agrobacterium tumefaciens KYLX71:35S² vector (Maiti et al. 1993) in both sense and antisense orientations, and transformed into tobacco and Arabidopsis. Northern analysis of the transgenic Arabidopsis and tobacco lines confirmed that AtMinE1 was being expressed in the transgenic lines (FIG. 9B). The level of expression observed in the Arabidopsis lines showed that the AtMinE1 transgene was overexpressed compared to the wild-type level except in line 2 where the level appeared to be only slightly more than in the wild type. It was observed that in both the transgenic Arabidopsis and tobacco lines two bands hybridized with similar intensity in the northern blots using the AtMinE1 probe (FIGS. 9B, C). Since no tobacco MinE homologue was observed to cross-hybridize to the AtMinE1 probe, and the AtMinE1 cDNA was used in the KYLX71 plant transformation vector, the two bands observed may be due to alternative processing in the pea rbcS3′ polyadenylation sequences (Mogen et al, Mol. Cell. Biol. 12:5406-5414 (1992)), although it is also possible that mRNA degradation occurred in the samples.

None of the tobacco plants overexpressing the antisense construct showed any phenotypic effect. No difference in chloroplast morphology was observed either using the confocal or electron microscope. This was expected since the AtMinE1 gene did not corss-hybridize to a tobacco homologue in the northern or Southern blots (FIG. 9C).

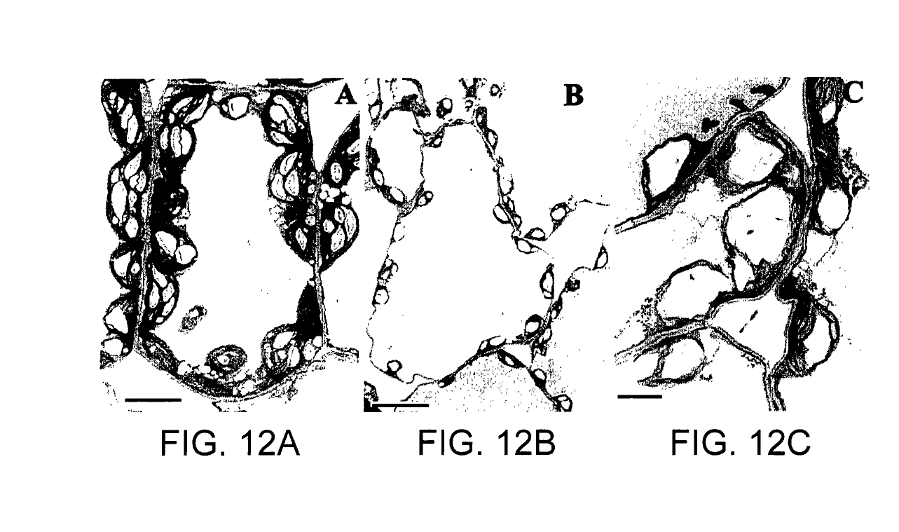

Arabidopsis plants expressing the antisense construct, on the other hand, appeared very small during selection on kanamycin-containing medium. Attempts to take these small plants out of tissue culture were not successful as none of the plants survived the transfer into soil. As chloroplasts from tissue-culture-grown plants contain more starch and the thylakoid membranes are more diffuse, tissues from tissue-culture-grown wild-type Arabidopsis and a transgenic line containing an auxin-response gene that does not affect the leaf or chloroplast morphology were compared. Electron microscopy of plants in culture revealed that the chloroplasts of these plants were very small with very few thylakoid membranes (FIGS. 12B, C).

We claim:

1. A vector comprising an exogenous gene which encodes a protein which has the same functional activity as a protein encoded by the Arabidopsis thaliana MinE or MinD gene and which when expressed in a plant cell causes the plant cell to have enlarged and/or a reduced number of chloroplasts.

2. A cell comprising the vector of claim 1.

3. A tissue culture comprising cells of claim 2.

4. A seed comprising the vector of claim 1.

5. The vector according to claim 1, wherein said exogenous gene is derived from Arabidopsis thaliana.

6. The vector according to claim 1, wherein the exogenous gene is an exogenous MinD gene.

7. The vector according to claim 6, wherein said exogenous MinD gene is derived from Arabidopsis thaliana MinD gene.

8. The vector according to claim 1, wherein the exogenous gene is an exogenous MinE gene.

9. The vector according to claim 8, wherein said exogenous MinE gene is derived from Arabidopsis thaliana MinE gene.

10. A transgenic plant comprising within its nuclear genome an exogenous gene, wherein said exogenous gene encodes a protein which has the same functional activity as a protein encoded by the Arabidopsis thaliana MinE or MinD gene and which when expressed in a plant cell causes the plant cell to have enlarged and/or a reduced number of chloroplasts.

11. The transgenic plant according to claim 10, wherein said exogenous gene is derived from Arabidopsis thaliana.

12. The transgenic plant according to claim 10, wherein said exogenous gene is an exogenous MinD gene.

13. The transgenic plant according to claim 12, wherein said exogenous MinD gene is derived from Arabidopsis thaliana MinD gene.

14. The transgenic plant according to claim 10, wherein said transgenic plant is a tobacco plant.

15. The transgenic plant according to claim 10, wherein said exogenous gene is an exogenous MinE gene.

16. The transgenic plant according to claim 15, wherein said exogenous MinE gene is derived from Arabidopsis thaliana MinE gene.

17. A method of transforming the chloroplast genome of a plant, said method comprising the steps of:
A) producing a nuclear transgenic plants which contains large chloroplasts by:
i) providing a vector comprising an exogenous gene which encodes a protein which has the same functional activity as a protein encoded by the Arabidopsis thaliana MinE or MinD gene and which when expressed in a plant cell causes the plant cell to have enlarged and/or a reduced number of chloroplasts; and,
ii) transforming the nuclear genome of a plant with said vector which comprises said exogenous gene; and,
B) transforming the chloroplast genome of said nuclear transgenic plant with a vector which comprises Gene of interest.

18. The method of claim 17, wherein said exogenous gene is derived from Arabidopsis thaliana.

19. The method of claim 17, wherein said exogenous gene is an exogenous MinD gene.

20. The method of claim 19, wherein said exogenous MinD gene is derived from Arabidopsis thaliana MinD gene.

21. The method of claim 17, wherein said nuclear transgenic plant is a tobacco plant.

22. A chloroplast transgenic plant produced by the method of claim 17.

23. The method of claim 17, wherein said exogenous gene is an exogenous MinE gene.

24. The method of claim 23, wherein said exogenous MinE gene is derived from Arabidopsis thaliana MinE gene.

25. A method of selecting for plants that are chloroplast transgenics but not nuclear transgenics, wherein said method comprises:
A) crossing a plant produced by the method of claim 22 with a wild-type plant; and,
B) segregating out the plants which express the exogenous gene or genes of interest in the chloroplast genome and further do not express the exogenous gene in the nuclear genome by identifying which plants have normal chloroplast size and number and have the desired characteristic produced by the exogenous gene expressed in the chloroplast genome.

26. The method of claim 25, wherein said exogenous gene is derived from Arabidopsis thaliana.

27. The method of claim 25, wherein said plant which is a chloroplast transgenic is a tobacco plant.

28. A method of producing a transgenic plant which comprises one or a few large chloroplasts, said method comprising the steps of:
A) producing a vector comprising an exogenous gene which encodes a protein which has the same functional activity as a protein encoded by the Arabidopsis thaliana MinE or MinD gene and which effects a plant cell by allowing for the expression of only one or a few large chloroplasts; and
B) transforming the nuclear genome of a plant with said vector.

29. The vector according to claim 28, wherein said exogenous gene is derived from Arabidopsis thaliana.

30. The vector according to claim 28, wherein the exogenous gene is an exogenous MinD gene.

31. The vector according to claim 30, wherein said exogenous MinD gene is derived from Arabidopsis thaliana MinD gene.

32. The vector according to claim 28, wherein the exogenous gene is an exogenous MinE gene.

33. The vector according to claim 32, wherein said exogenous MinE gene is derived from Arabidopsis thaliana MinE gene.
